(19) United States
(12) Patent Application Publication
(10) Pub. No.: US 2015/0284018 A1
(43) Pub. Date: Oct. 8, 2015

(54) CONTAINMENT DEVICE AND TRANSPORT SYSTEM FOR REUSABLE MEDICAL DEVICE STERILIZATION, STORAGE, AND TRANSPORT

(71) Applicant: STERI SC INTERNATIONAL LLC, Beverly Hills, CA (US)

(72) Inventor: Mark D. Krosney, South Glastonbury, CT (US)

(21) Appl. No.: 14/680,333

(22) Filed: Apr. 7, 2015

Related U.S. Application Data
(60) Provisional application No. 61/976,458, filed on Apr. 7, 2014.

(51) Int. Cl. B62B 5/00; A61L 2/07

(52) U.S. Cl. CPC B62B 5/0026; A61L 2/07; B62B 5/0083

(57) ABSTRACT

A transport system for transporting a containment device used for reusable medical device sterilization features a vertically adjustable platform and a hinged autoclave interface.

### CROSS-REFERENCE TO RELATED APPLICATIONS

This application is a non-provisional of, and claims priority to, U.S. Patent Application Ser. Nos. 61/976,458, filed on Apr. 7, 2014; which application is incorporated in its entirety as if fully set forth herein.

### STATEMENT REGARDING FEDERALLY SPONSORED RESEARCH OR DEVELOPMENT

Not applicable.

### FIELD OF THE INVENTION

This invention relates generally to the field of medical equipment, devices, and systems; and, more specifically, to containment devices and transport systems for reusable medical device sterilization.

### BACKGROUND

The U.S. Food and Drug Administration identifies a sterilization wrap as “a device intended to be used to enclose another medical device that is to be sterilized by a health care provider,” and “is intended to allow sterilization of the enclosed medical device and also to maintain sterility of the enclosed device until used.” (See 21 CFR 880.6850.) The main purposes of a containment device are to “organize, permit sterilization of and sterilant removal from, protect, and safely store the instrumentation within.” (See Association for the Advancement of Medical Instrumentation, Containment devices for reusable medical device sterilization, AAMI FDS/ST77:2006/(R)2010.) Additionally, some containment devices maintain the sterility of the contents after sterilization until the point of use. (Id.) Sterilization wraps and containment devices come in the form of many different packaging systems such as woven textiles, non-woven materials, and rigid sterilization containers. Reusable rigid sterilization containers are the most durable option as they are constructed of rigid materials such as metal, plastic, or composite materials. These containment devices serve as packaging for instruments and other medical devices before, during, and after the sterilization process.

“Metal and plastic reusable rigid sterilization containers for surgical instruments have been in use in the United States for more than 25 years.” (Id.) In some cases, self-contained, reusable rigid sterilization containers include a barrier system, such as filters or valves. It is known in the art to utilize rigid sterilization containers including a filtered base and a lockable filtered lid. This type of system generally consists of a bottom or base with carrying handles and a lid that is secured to the base by means of a latching mechanism. A basket or tray to hold instruments or other items to be sterilized is placed inside. A filter or valve system is incorporated into the lid and/or base to provide for air evacuation and sterilant penetration during the sterilization cycle and to act as a barrier to microorganisms during storage, handling, and transport.

Due to handling requirements, as well as industry standards, the combined weight of the containment device, the instruments, and any accessories or wrappers shall not exceed 25 pounds, thereby necessitating multiple containers for many types of surgical procedures.

In other cases, reusable rigid sterilization containers are capable of accepting multiple trays of reusable medical devices, such as a vented rigid cabinet with gasketed door(s) configured to seal against the cabinet opening.

Disposable filter material is held in place over the cabinet vent openings by a filter retainer that is attached by threaded studs and fasteners. Similar to the rigid sterilization containers with lids, as described above, this sterilization cabinet is loadable into an autoclave such as the industrial autoclaves used in hospitals, where the cabinet, and the load inside, are sterilized. The filtered vents and the sealed door(s) are intended to provide aseptic presentation of the now sterilized surgical instruments in the operating room.

An autoclave is a sterilization chamber holding items for sterilization, such as medical surgical instruments. A sterilant is introduced into the sealed chamber. A sterilant is an agent used to destroy microorganisms such as disease-causing pathogens. Sterilant agents can be chemical, or non-chemical, such as radiation and very high temperatures.

Still other instances of the prior art disclose containment devices that serve as self-contained autoclaves, allowing sterilization of the interior of the device and its contents. The prior art further discloses a transfer cart as well as a modular lift device for stacked storage of the device.

Information relevant to attempts to address the problems found in the current state of the art, can be found in U.S. Pat. No. 8,454,901, and U.S. patent application Ser. No. 13/944,875, published on Jan. 22, 2015 as United States Patent Publication Number 2015/0023839 A1. However, each one of these references suffers from one or more of the following disadvantages:

a) a fixed height transfer cart makes it difficult to load and unload a cabinet in an ergonomically responsible, convenient and sterile manner;

b) fixed height transfer carts do not adequately maintain aseptic presentation of the sterilized contents since the sterile field varies from person to person;

c) transporting cabinet-style containment devices through hospital corridors is difficult because of partially blocked sight;

d) locking wheels on previously disclosed transfer carts are difficult to actuate and not failsafe;

e) the weight of a fully loaded cabinet may make it difficult to maneuver the cabinet up or down inclines or across uneven surfaces;

f) the wheels on previously disclosed transfer carts are too small for variations in floor surfaces and elevator entrances;

g) delicate, sterilized instruments and equipment may impinge on one another during transport causing damage or questionable operability due to rattling and instability of rigid transfer cart over uneven surfaces;

h) the autoclave interface adapter protrudes from the transfer cart, which is space-consuming and a safety hazard;

i) as disclosed in the prior art, the mating frame for the gasket seal in the cabinet access means has a sharp edge upon which the instruments or the appendages of the various hospital staff could catch and cause damage or injury;

j) the disclosed gasket interface, between the cabinet frame and cabinet access means, does not provide a microbial barrier that prevents recontamination of the containment device contents once they are sterilized since breaking the gasketed seal in the prior art cabinet-style containment devices causes unfiltered air from outside of the sterile field to rush into the cabinet interior;

k) shelf support system has numerous sharp edges and prominences which may damage instruments or injure staff;

l) latching mechanisms must ensure a secure gasket seal as part of the microbial barrier, permitting opening of the reusable rigid sterilization container without compromising aseptic presentation of the sterile contents, as disclosed, and with respect to the cabinet access means, the multiple latches require manual alignment and two hands for operation which is prone to error and compromising the aseptic presentation of the sterile contents;

m) present door geometry requires valuable space in an operating room since the current door arc geometry is required to maintain the sterile field;

n) no visibility is available to the contents of the cabinet to ensure accurate inventory and identification of sterile components;

o) the filter and filter retention means are not adequately evacuating during vacuum cycle resulting in the potential for recontamination since moisture creates a pathway for microorganisms to enter the containment device and re-contaminate the instruments inside;

p) filter installation is tedious and requires full opening of access door frame in top and bottom of cabinet;

q) installation of unsupported sheet filter material is susceptible to misplacement, creasing, or tearing which defeats the purpose of the filter as a microbial barrier, which is critical in order to prevent recontamination of the containment device contents once they are sterilized;

r) as disclosed, cabinet-style containment devices are monolith, welded structures that are difficult to manufacture, requiring many processes, and a late cycle error causes expensive, risky repair, furthermore, the welded cabinet structure is heavy and difficult to handle and transport; and

s) current cabinet-style containment devices are manufactured of stainless steel which is heavy and has poor thermal conductivity.

It would, therefore, be desirable to have a containment device and transfer system for reusable medical device sterilization that meets or exceeds FDA requirements for reusable medical device sterilization, and addresses the above-stated shortcomings of the known art.

### SUMMARY OF THE INVENTION

In this specification and in the appended claims and drawings, words and phrases have the meanings commonly attributed to them in the relevant art except as otherwise specified herein.

In this specification and in the appended claims and drawings, “sterile field,” including grammatical equivalents, singular and plural, is an area that has been prepared for surgical procedure and is considered free of microorganisms. With respect to surgical staff responsible for removing sterilized medical equipment from a containment device, the sterile field is defined as the area in front of the staff person, from just below their neckline to just above the bottom of their gown.

In this specification and in the appended claims and drawings, “health care facility,” including grammatical equivalents, singular and plural, means hospitals, nursing homes, extended-care facilities, free-standing surgical center, clinics, and medical and dental offices. For convenience, the term “hospital” is sometimes used and shall carry the same meaning.

In this specification and in the appended claims and drawings, “wrap,” including grammatical equivalents, singular and plural, means a containment device for reusable medical device sterilization. The “wrap” may be flexible or rigid. The terms “sterilization wrap,” “sterilization container,” “containment device,” and equivalents, are to carry the same meaning and may be used interchangeably in this specification and in the appended claims and drawings.

In this specification and in the appended claims and drawings, “aseptic presentation,” including grammatical equivalents, singular and plural, means maintaining the sterility of the contents as a sterilized package or containment device is opened and the contents are removed.

In this specification and in the appended claims and drawings, “biological indicator” (BI), including grammatical equivalents, singular and plural, means a test system containing viable microorganisms providing a defined resistance to the specified sterilization process.

In this specification and in the appended claims and drawings, “chemical indicator” (CI), including grammatical equivalents, singular and plural, means a test system that reveals change in one or more predefined process variables based on a chemical or physical change resulting from exposure to a process.

In this specification and in the appended claims and drawings, “cycle, steam sterilization, dynamic-air-removal type,” including grammatical equivalents, singular and plural, refers to one of two types of sterilization cycles in which air is removed from the chamber and the load by means of a series of pressure and vacuum excursions (pre-vacuum cycle) or by means of a series of steam flushes and pressure pulses above atmospheric pressure (steam-flush pressure-pulse [SFPP] cycle). In pre-vacuum steam sterilizers, the dynamic-air-removal cycle depends upon one or more pressure and vacuum excursions at the beginning of the cycle to remove air. This method of operation results in shorter cycle times for wrapped items because of the rapid removal of air from the chamber and the load by the vacuum system and because of the usually higher operating temperatures (132° C. to 135° C. [270° F. to 275° F.]). This type of cycle generally provides for shorter exposure times and accelerated drying of fabric loads by pulling a further vacuum at the end of the sterilizing cycle. In steam-flush pressure-pulse steam sterilizers, the dynamic-air-removal cycle depends upon a repeated sequence consisting of a steam flush and a pressure pulse to remove air from the sterilizing chamber and processed materials. As is the case with pre-vacuum sterilizers, the dynamic-air-removal cycle of a steam-flush pressure-pulse sterilizer rapidly removes air from the sterilizing chamber and wrapped items. Air removal is achieved with the sterilizing chamber pressure at above atmospheric pressure (no vacuum is required to remove air for sterilization). Typical operating temperatures are 132° C. to 135° C. (270° F. to 275° F.).

In this specification and in the appended claims and drawings, “cycle, steam sterilization, gravity-displacement type,” including grammatical equivalents, singular and plural, refers to a type of sterilization cycle in which incoming steam displaces residual air through a port or drain in or near the bottom (usually) of the sterilizer chamber. Typical operating temperatures are 121° C. to 123° C. (250° F. to 254° F.) and 132° C. to 135° C. (270° F. to 275° F.).

In this specification and in the appended claims and drawings, “load,” including grammatical equivalents, singular and plural, means similar items requiring the same sterilization parameters that are sterilized together.

In this specification and in the appended claims and drawings, “sterile barrier system,” including grammatical equivalents, singular and plural, means minimum packaging configuration that maintains sterility of the package contents until aseptic presentation at the point of use.

The present invention addresses one or more of the problems and deficiencies of the known art discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

In view of the foregoing, an embodiment herein provides a novel containment device and transport system for reusable medical device sterilization. Specifically, an embodiment of the present invention includes a transport apparatus for a sterilization cabinet. The transport apparatus, or transport cart, includes a base structural frame, a vertically adjustable platform configured to accept the sterilization cabinet, a hinged sterilization chamber interface, and a handle.

Briefly, another embodiment of the present invention includes a rigid containment device, or sterilization cabinet, with at least one vent, where the rigid containment device is configured to accept a load for sterilization.

The containment device includes at least one filter apparatus configured to be sealably secured to be coincident with the at least one vent. An aperture in the containment device sealably accepts at least one door with a viewing window.

According to another embodiment of the present invention, a filter system for a sterilization cabinet includes at least one filter apparatus securably sealed to a vent in the sterilization cabinet. The filter apparatus includes: at least one layer of steam-permeable filtration material, a support frame, a filter retainer, a linkage mechanism connecting the support frame to the filter retainer, an integral seal, and a single use indicator.

### BRIEF DESCRIPTION OF THE DRAWINGS

These and other features, aspects, and advantages of the present invention will become better understood with regard to the following description, appended claims, and accompanying drawings where:

FIG. 1 illustrates an exploded view of a containment device according to an embodiment herein;

FIG. 2 illustrates an assembled view of a containment device according to an embodiment herein;

FIG. 3 illustrates a view of a containment device aperture seal according to an embodiment herein;

FIG. 4 illustrates a view of a containment device in an open state according to an embodiment herein;

FIG. 5 illustrates a view of a containment device in a closed state according to an embodiment herein;

FIG. 6 illustrates a hidden-line view of the interior configuration of a containment device in a closed state according to an embodiment herein;

FIG. 7 illustrates a view of a containment device aperture seal (door or latch) in a closed state according to an embodiment herein;

FIG. 8 illustrates a view of a containment device aperture seal (door or latch) in a partially open state according to an embodiment herein;

FIG. 9 illustrates a view of a containment device aperture seal (door or hatch) in a fully open state according to an embodiment herein;

FIG. 10 illustrates a view of a containment device aperture seal (door or hatch) in a closed state according to an embodiment herein;

FIGS. 11 and 12 illustrate views of a containment device aperture seal (door or hatch) in a partially open state according to an embodiment herein;

FIG. 13 illustrates a view of a containment device aperture seal (door or hatch) in a fully open state according to an embodiment herein;

FIG. 14 illustrates a view of a containment device aperture seal (door or hatch) in a closed state according to an embodiment herein;

FIG. 15 illustrates a view of a containment device aperture seal (door or hatch) in a partially open state according to an embodiment herein;

FIG. 16 illustrates a view of a containment device aperture seal (door or hatch) in a fully open state according to an embodiment herein;

FIG. 17 illustrates a view of a door latch mechanism according to an embodiment herein;

FIG. 18 illustrates a view of a door latch mechanism according to an embodiment herein;

FIG. 19A, FIG. 19B, and FIG. 19C illustrate three positions of a door latch mechanism according to an embodiment herein;

FIG. 19A illustrates the door latch mechanism in the latched and locked position, according to an embodiment herein;

FIG. 19B illustrates the door latch mechanism in the unlocked position, according to an embodiment herein;

FIG. 19C illustrates the door latch mechanism in the unlatched position, according to an embodiment herein;

FIG. 20A illustrates a sectional view of a door latch mechanism in the locked position, according to an embodiment herein;

FIG. 20B illustrates a sectional view of a door latch mechanism in the unlocked position, according to an embodiment herein;

FIG. 21A illustrates the horizontal support surface with the pins engaged, according to an embodiment herein;

FIG. 21B illustrates the horizontal support surface with the pins retracted, according to an embodiment herein;

FIG. 22 illustrates a view of a vented horizontal support surface with extensible slides according to an embodiment herein;

FIG. 23 illustrates a view of a filter apparatus according to an embodiment herein;

FIG. 24 illustrates a detail cross-sectional view of a filter apparatus according to an embodiment herein;

FIG. 25 illustrates an exploded view of a filter apparatus according to an embodiment herein;

FIG. 26A illustrates the filter apparatus frame, according to an embodiment herein;

FIG. 26B illustrates a section of the filter apparatus frame showing a hole through which a post is introduced, according to an embodiment herein;

FIG. 26C illustrates the section of FIG. 26B with the post protruding through the hole, according to an embodiment herein;

FIGS. 27A through 27C illustrates views of a filter system according to an embodiment herein;

FIG. 27A illustrates the filter apparatus before placement in the filter retainer, according to an embodiment herein;

FIG. 27B shows the filter apparatus positioned within the open filter retainer, according to an embodiment herein;

FIG. 27C shows the filter apparatus positioned within the closed filter retainer, according to an embodiment herein;

FIG. 28A illustrates a view of a filter system incorporated into the roof and floor of an open containment device, according to an embodiment herein;

FIG. 28B illustrates a view of a filter apparatus being loaded into the filter retainer of a closed containment device, according to an embodiment herein;

FIG. 29A illustrates a detailed sectional side view of the filter system in the closed position, according to an embodiment herein;

FIG. 29B illustrates a detailed sectional side view of the filter system in the open position, according to an embodiment herein;

FIGS. 30-35 illustrate views of a transport cart according to an embodiment herein;

FIG. 30 illustrates a view of a transport cart with a basket, according to an embodiment herein;

FIG. 31A illustrates a view of the transport cart of FIG. 30 with the autoclave interface adapter in the stowed position, according to an embodiment herein;

FIG. 31B illustrates the transport cart of FIG. 31A with the autoclave interface adapter in the extended position, according to an embodiment herein;

FIG. 32A illustrates a view of the cabinet loading surface of the transport cart in the lowest vertical position, according to an embodiment herein;

FIG. 32B illustrates a view of the vertical adjustment mechanism used to raise the cabinet loading surface, according to an embodiment herein;

FIG. 33A illustrates a view of the transport cart with the handle in the locked position, according to an embodiment herein;

FIG. 33B illustrates a view of the transport cart with the handle in the unlocked position, according to an embodiment herein;

FIG. 34A illustrates a side view of the autoclave interface adapter, according to an embodiment herein;

FIG. 34B illustrates an oblique view of the autoclave interface adapter, according to an embodiment herein;

FIG. 35 illustrates a view of the transport cart with sensing apparatus, according to an embodiment herein;

FIG. 36 illustrates a containment device and transfer system for reusable medical device sterilization, storage, and transport according to an embodiment herein.

### DETAILED DESCRIPTION OF THE INVENTION

In the Summary of the Invention above, in the Description and appended Claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

The term “at least” followed by a number is used herein to denote the start of a range beginning with that number (which may be a range having an upper limit or no upper limit, depending on the variable being defined). For example “at least one” means one or more than one. The term “at most” followed by a number is used herein to denote the end of a range ending with that number (which may be a range having one or zero as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

While the specification will conclude with claims defining the features of embodiments of the invention that are regarded as novel, it is believed that the invention will be better understood from a consideration of the following description in conjunction with the figures, in which like reference numerals are carried forward.

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

In this specification, where a document, act, or item of knowledge is referred to or discussed, this reference or discussion is not an admission that the document, act, or item of knowledge or any combination thereof was, at the priority date, publicly available, known to the public, part of common general knowledge, or otherwise constitutes prior art under the applicable statutory provisions; or is known to be relevant to an attempt to solve any problem with which this specification is concerned.

We discuss a cabinet-style containment device configured to accept at least one sterilization tray, that addresses one or more of the above-stated ergonomic and safety concerns. To that end, we combine at least one containment device with at least one transport cart for safe and ergonomic transport and aseptic presentation of the sterilized load, that incorporates a vertical adjustment mechanism as part of the transport cart for height adjustment while loading and unloading into the sterilization chamber such as an autoclave, storage, transporting, and loading and unloading of contents while maintaining aseptic presentation of the load by adjusting to each user's sterile field.

Embodiments of the present invention, as described here, and in the appended claims and drawings, include, but are not limited to, containment devices for reusable medical device sterilization and storage, filter apparatus, transport carts for transport, storage, and sterilization of containment devices, and a system for sterilizing, storing, and transporting reusable medical devices.

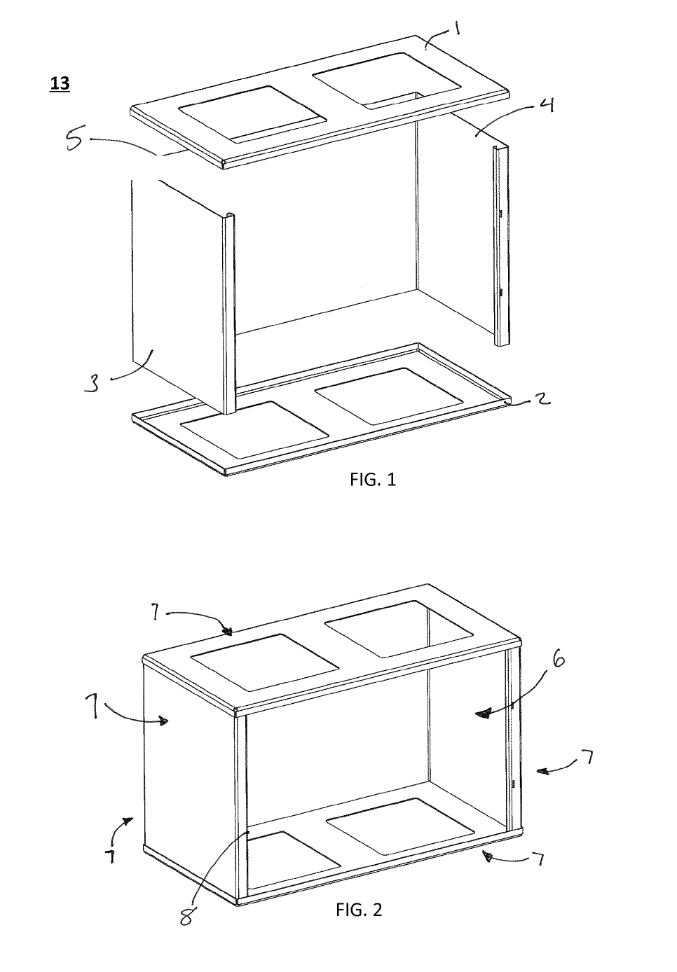

Referring now to the drawings in general and to FIG. 1 in particular, there is shown an illustration of a containment device 13 for reusable medical device sterilization in accordance with an embodiment of the present invention. The containment device 13 of FIG. 1 includes: a roof 1, a floor 2, a left side 3, a right side 4, and a back side 5. FIG. 2 shows a containment device interior 6, a containment device exterior 7, and a containment device aperture 8.

The containment device interior 6 is configured to accept a load for sterilization. The sterilization load can include, for example, reusable medical instruments. The containment device aperture 8 is configured to sealably accept an at least one containment device door or hatch. The roof 1 and floor 2 may each be configured to accept an at least one filter apparatus.

Referring now to FIG. 2, in embodiments of the containment device 13, the roof 1, floor 2, left side 3, right side 4, and back side 5 may be joined employing any of a number of manufacturing methods including, but not limited to, continuous welded seams, spot welding with additional sealing material interposed between adjoining sides, or by using mechanical fasteners with additional sealing material interposed between adjoining sides. The various joining methods available are employed so as to create a joint that acts as a barrier to prevent the escape of the sterilant, even under various temperature and humidity cycling conditions.

In one embodiment of the containment device 13, the roof 1 is manufactured as a panel with a substantially flat surface portion and a formed lip extending perpendicular to the flat surface portion forming a continuous perpendicular surface around the perimeter of the flat surface. The flat surface portion is configured to accept a filter apparatus. Embodiments may include a configuration where the flat surface portion comprises at least one void area, each void area accepting of a filter system.

In one embodiment of the containment device, the floor 2 is substantially similar to the roof 1, with a flat surface portion, a perimeter lip, and at least one void area, except in that it is assembled “upside down” with respect to the roof.

In one embodiment of the containment device 13, the left side 3, right side 4, and back side 5 are a single formed panel with a top edge and a bottom edge, the top edge configured to sealably accept the roof 1, and the bottom edge configured to sealably accept the floor 2.

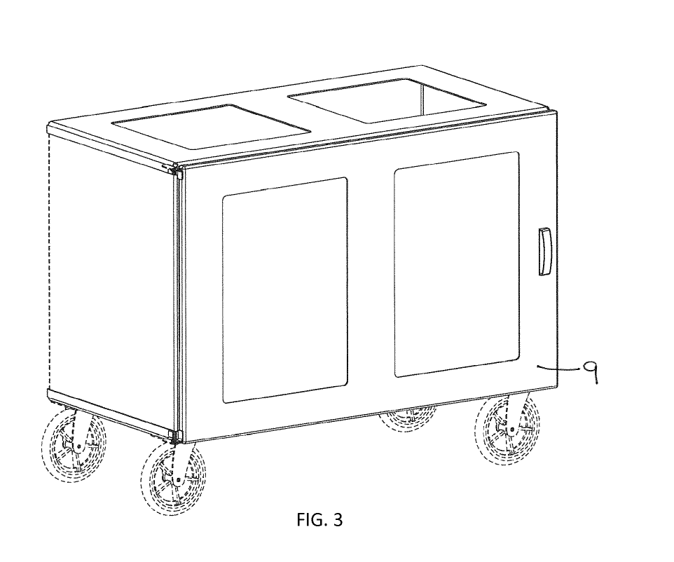

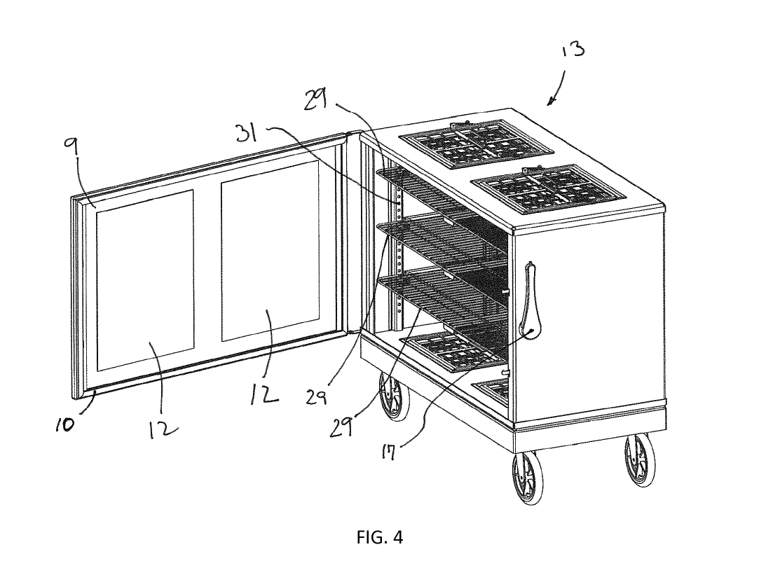

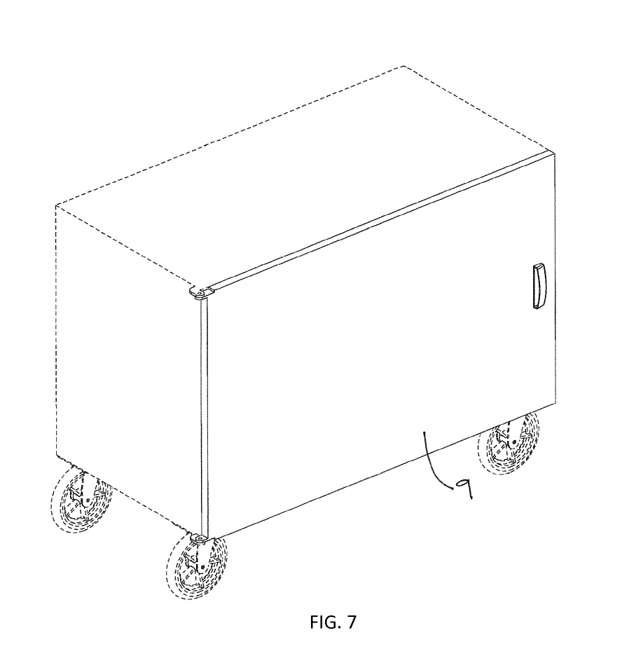

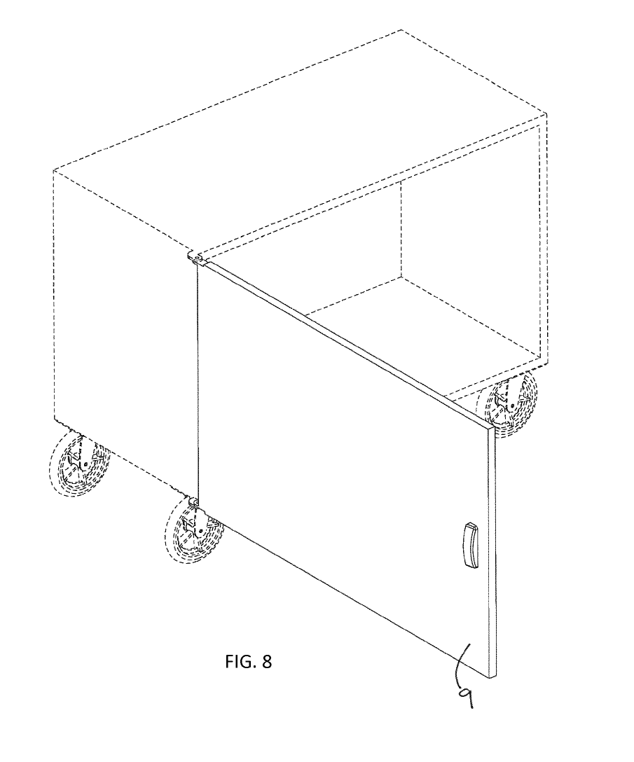

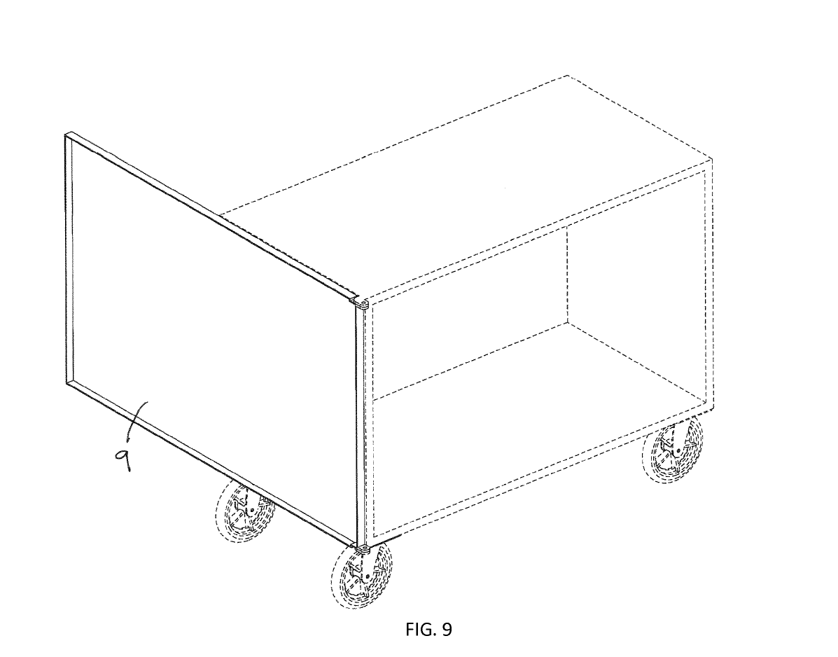

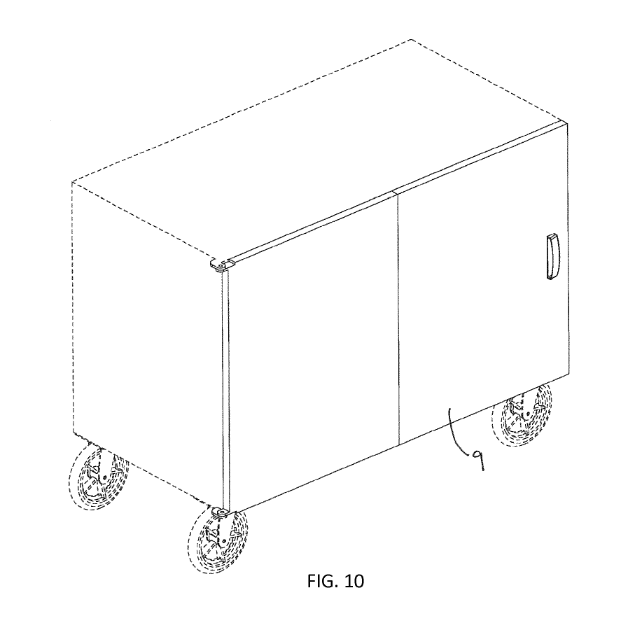

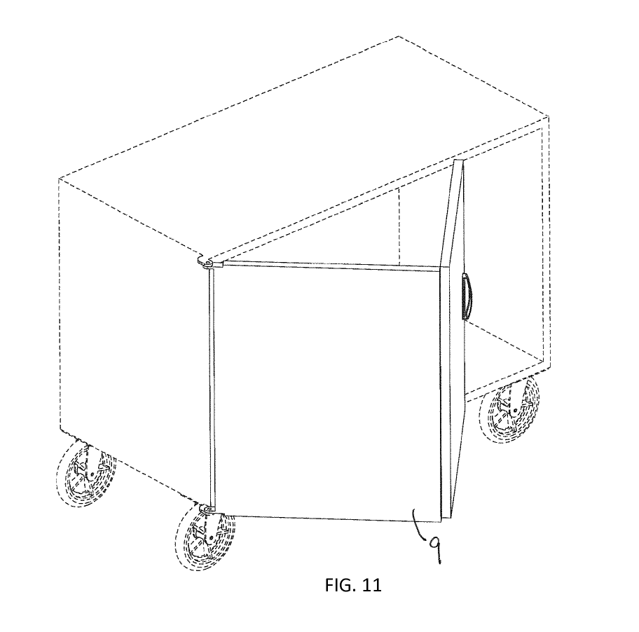

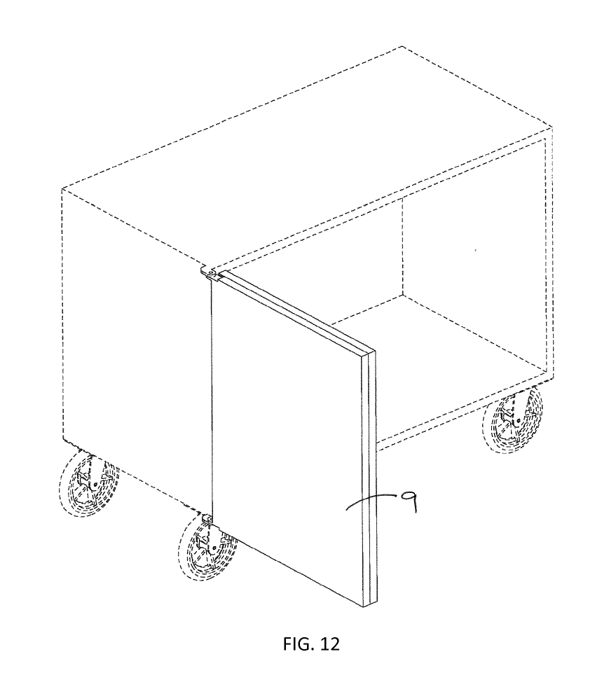

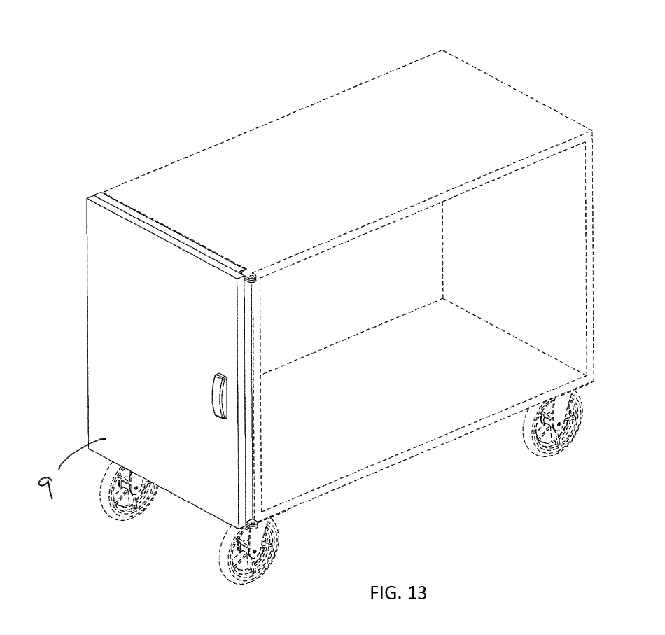

In embodiments of the containment device 13 shown in FIG. 3, the aperture 8 defined by the roof, floor, left side, and right side is configured to accept a containment device aperture seal 9. The containment device aperture seal 9 as shown in FIG. 4 may typically be a door or hatch 9 with an integral gasket 10 configured to prevent escape of the sterilant during the sterilization cycle and to prevent microbial intrusion after the sterilization cycle, although one having ordinary skill in the art will recognize that the containment device aperture seal 9 may be accomplished by employing various structures such as, but not limited to, a one or more hinged door shown in FIGS. 7, 8, and 9, a one or more bi-fold door shown in FIGS. 10, 11, 12, and 13, a one or more fastened hatch, a one or more membrane barrier, or a one or more tracked door.

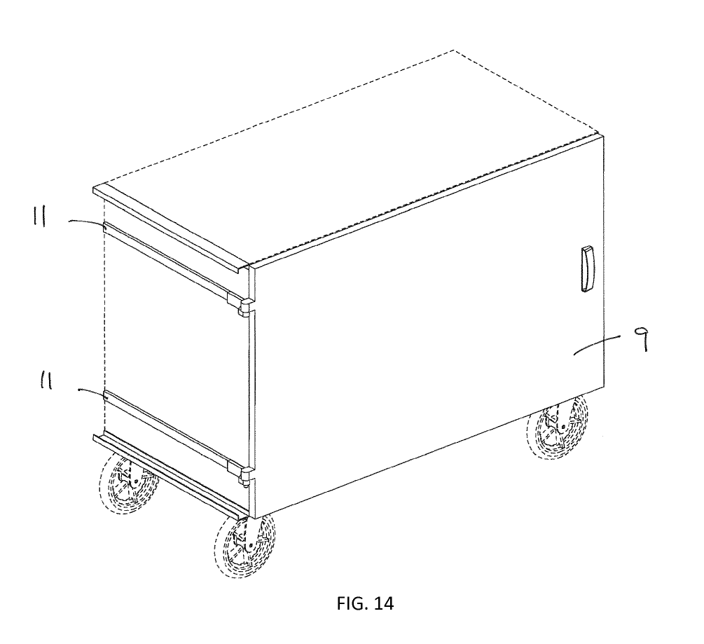

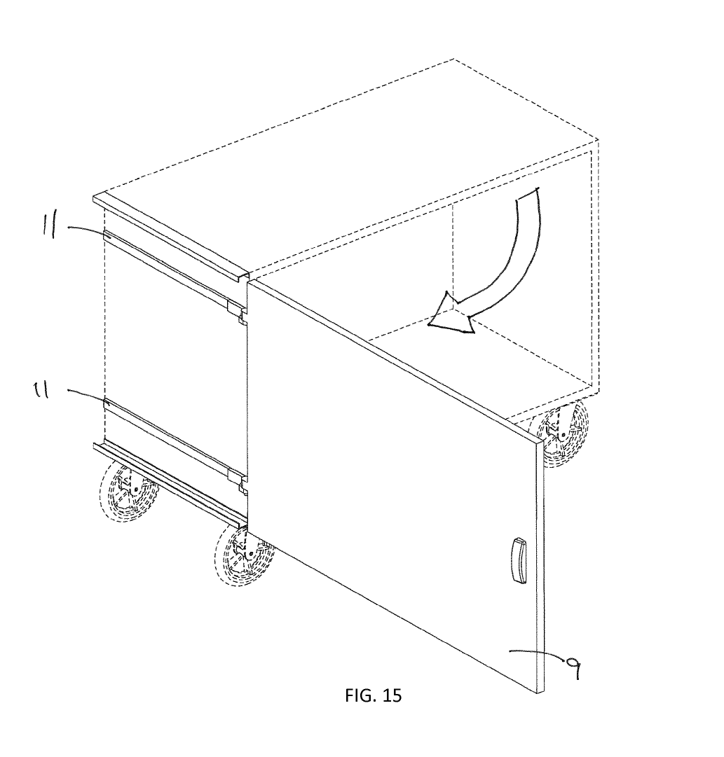

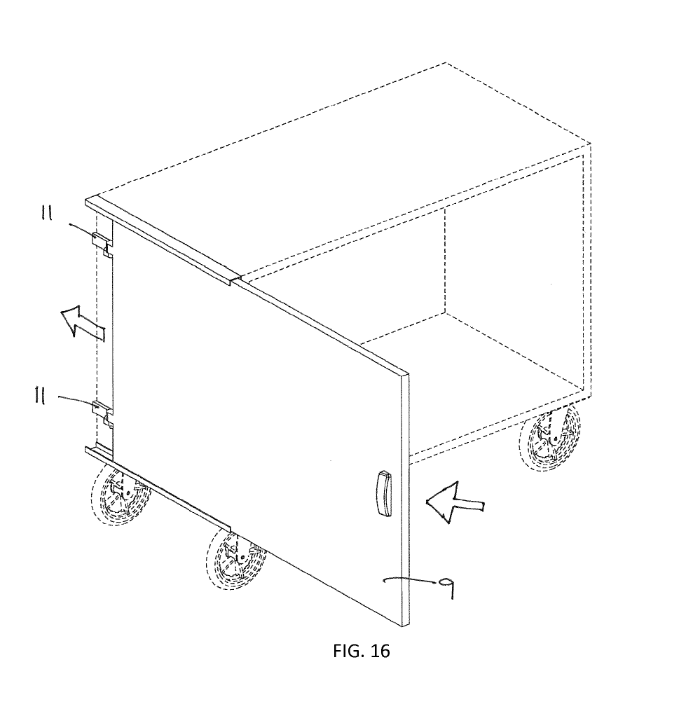

Embodiments of the containment device aperture seal 9 may combine one or more of the structures of FIGS. 14, 15, and 16, where for example, a hinged door 9 may be mounted onto tracks 11, or slides, such that the door 9 may, first, swing open then, second, slide back away from the operator thereby recovering usable space in front of and around the containment device 13 that would otherwise have been occupied by the door structure.

In a further embodiment of the containment device 13, an aperture seal 9 is in the form of a door or hatch as shown in FIG. 4. The door or hatch 9 may further include at least one viewing window 12. The viewing window 12 offers an improvement over the known art by allowing for the easy identification and inventory of the surgical instruments before, during, and after the sterilization process.

The at least one viewing window 12 may be made of a material that can accommodate for thermal expansion and other environmental stresses involved in a sterilization cycle. The interface between the door or hatch 9 and the at least one viewing window 12 may be configured to accommodate the thermal expansion of the various materials involved, as well as to prevent escape of the sterilant during the sterilization cycle and to prevent microbial intrusion after the sterilization cycle. The interface between the door or hatch 9 and the at least one viewing window 12 may be configured such that it discourages condensate from collecting or being trapped during and after the sterilization cycle.

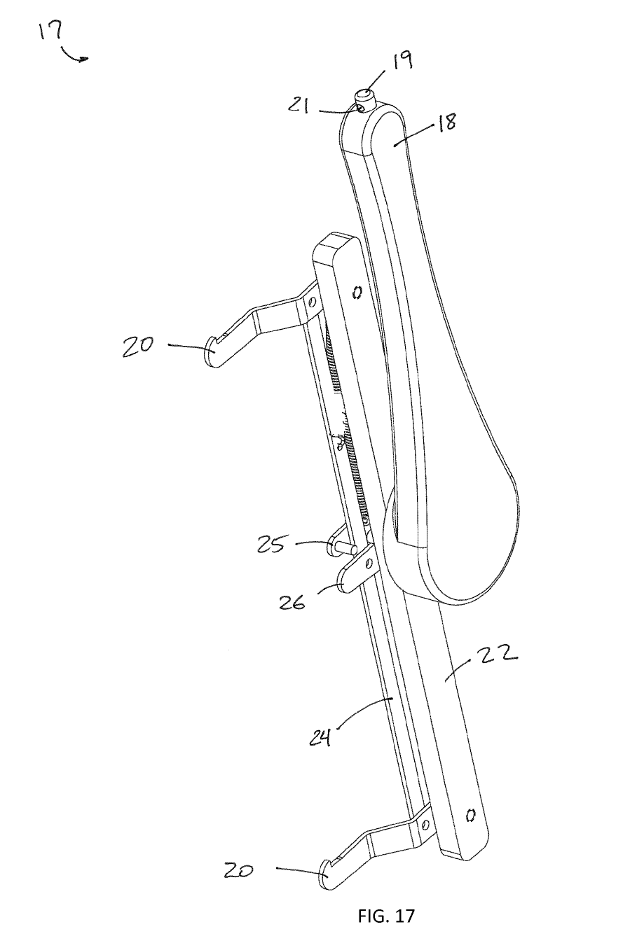

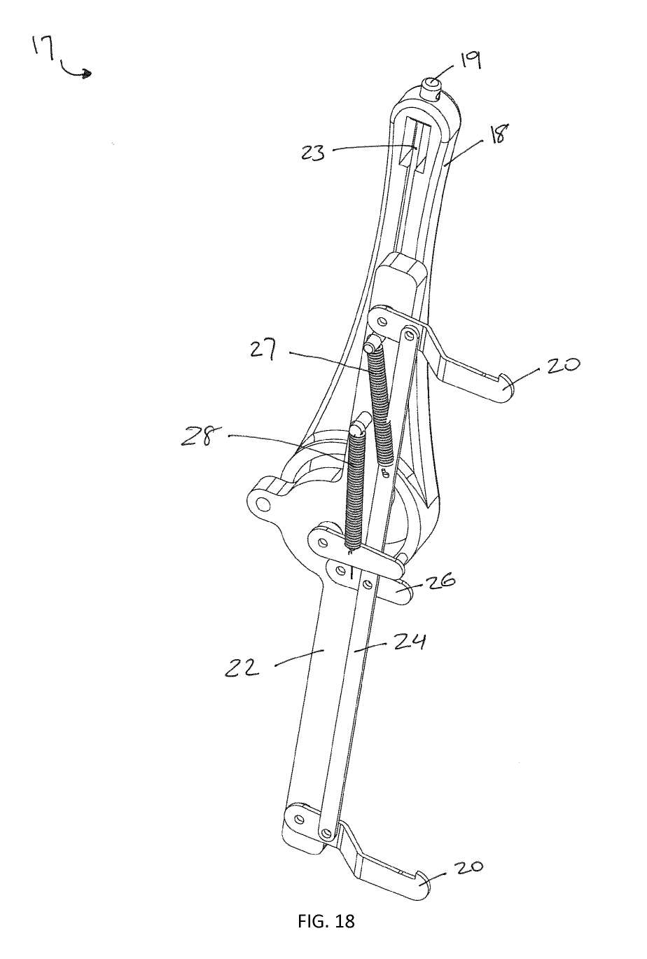

In embodiments of the containment device 13 shown in FIG. 4, the containment device aperture seal 9 may be retained in a closed, or sealed, condition with respect to the containment device 13 such that, while in the closed condition, sterilant may not escape from the containment device 13 during a sterilization cycle and, after the sterilization cycle, microbial intrusion into the interior 6 of the device 13 is blocked, by engaging an at least one door latch mechanism 17. Embodiments shown in FIGS. 17 and 18 may be configured such that a door latch mechanism 17 includes a latch arm 18, a lock button 19, and a one or more latching hooks 20 configured to interface with the containment device aperture seal 9.

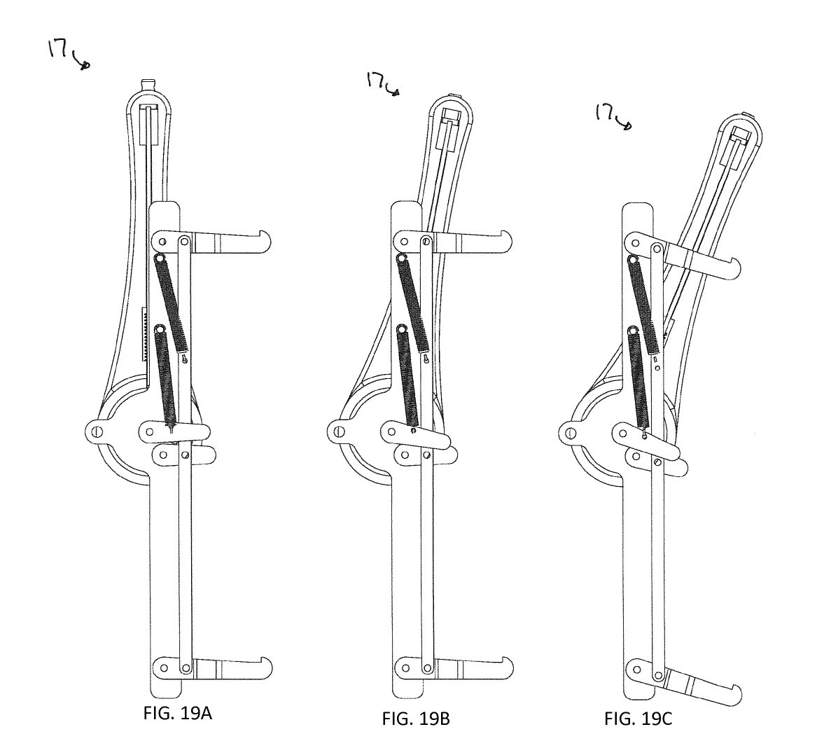

An embodiment of the door latch mechanism 17 can allow for one-handed operation, as shown in FIG. 19. In such a one-handed operation, the door latch mechanism 17 includes an arm 18 with a lock button 19. The lock button 19, which may further include features 21 to accommodate disposable safety locks, keeps the arm 18 locked to avoid accidental opening of the containment device 13. Once the lock button is depressed, the arm 18 is free to rotate. The rotating motion of the arm 18 disengages the one or more latches 20. In a further embodiment, the rotating motion of the arm 18 also pushes on the containment device seal 9 to break the seal and present the load.

In an embodiment of the door latch mechanism 17, the rotation of the arm 18 actuates a two-part action where, in the first action, a filtered vent is opened in order to equalize the air pressure between the inside of the device 13 and outside environment. Once this pressure is equalized, the arm 18 is free to continue to the second action, which releases the one or more latches from the containment device aperture seal 9, allowing for the seal 9 to be broken and providing for the aseptic presentation of the load. This two-part action avoids “air-rush” into the containment device 13 from outside of the sterile field, which may compromise the aseptic presentation of the load.

Embodiments of the door latch mechanism 17 may include a mount plate 22, an arm 18 pivotably attached to the mount plate 22, a lock button 19 slidingly mounted in the arm 18 wherein the lock button 19 includes an inspection tag hole 21, a lock plate 23 with a first end fixedly attached to the lock button 19 and a second end slidingly communicative with a mount plate lock detent 24.

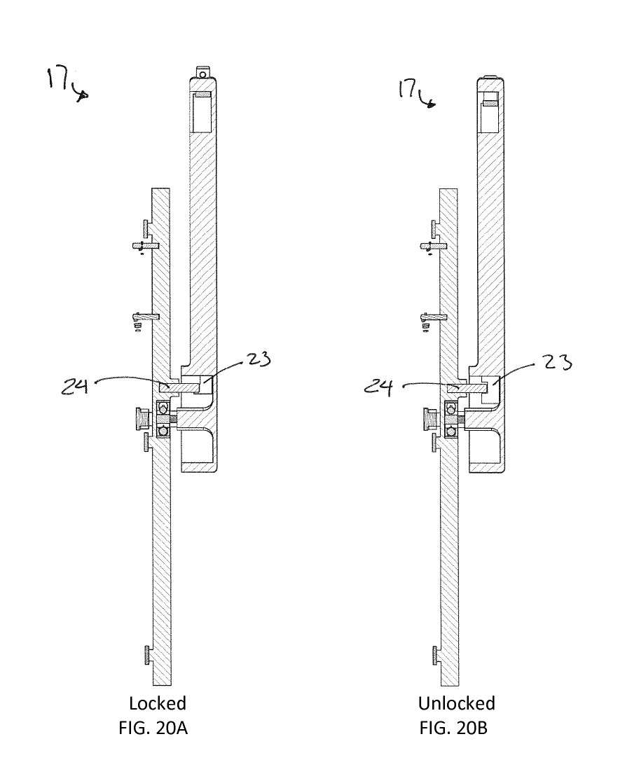

Referring to FIGS. 20A and 20B, the lock button 19 further includes one or more latches 20 pivotably attached to the mount plate 22, a link 24 pivotably connected to the one or more latches 20, a pusher plate 25 fixedly attached to the arm 18, an actuator plate 26 pivotably attached to the mount plate 22, a latch return spring 27 with a first end attached to the mount plate 22 and a second end attached to the link 24, and an arm return spring 28 with a first end attached to the mount plate 22 and a second end attached to the actuator plate 26.

Other embodiments of the door latch mechanism 17 may be configured such that the one or more door latches do not protrude from the containment device 13 until the door latch mechanism 17 is engaged. This embodiment would ensure that the containment device 13 is free from exposed latches that may catch on hospital equipment or personnel, surgical gowns, and the like.

Referring to FIG. 4, in embodiments of the containment device 13, the interior 6 of the device 13, as defined by the roof 1, the floor 2, the left side 3, the back side 5, the right side 4, and the vertical plane formed by the aperture (as defined above) 8, may include at least one vented horizontal support surface 29 with at least one integral retractable support pin 30, shown in FIGS. 21A and 21B, and at least one support receptacle 31 configured to receive the at least one integral retractable support pin 30.

The at least one vented horizontal support surface 29 can be sized to support surgical trays for sterilization. An embodiment may comprise a plurality of support receptacles 31 arranged in a vertical distribution between the floor and the roof such that the vertical positioning of the vented horizontal support surface may be adjusted by inserting the at least one retractable support pin into the desired support receptacle corresponding to the vertical position desired.

Referring to FIG. 19, in an embodiment of the containment device 13, the horizontal support surface 29 may comprise a vented planar surface 32 with a top face, a bottom face, a front edge, a back edge, a left edge and a right edge. In this embodiment, the planar surface 32 is vented with apertures that extend from the top face to the bottom face. The horizontal support surface 29 may further comprise a plurality of retractable lock pins 30, at least one retractable lock pin protruding from the left edge and at least one retractable lock pin protruding from the right edge. In some embodiments, the at least one retractable lock pin may protrude from the front edge or the back edge. The at least one retractable lock pin is configured to be retractable towards the edge from which it protrudes by a retractable lock pin release mechanism 33, preferably located in proximity to the center of the front edge. The retractable lock pins are configured to be biased “out” (in the “engaged” position) (see FIG. 21A) until such time as the retractable lock pin release mechanism is actuated (see FIG. 21B).

It is contemplated that the disclosed release mechanism and retractable lock pin system may be accomplished in many ways that are within the scope and spirit of this specification, as well as the appended claims and drawings. For example, the release mechanism may be accomplished via a spring-loaded cable-and-pulley system, or electronically via a one or more servo-motors.

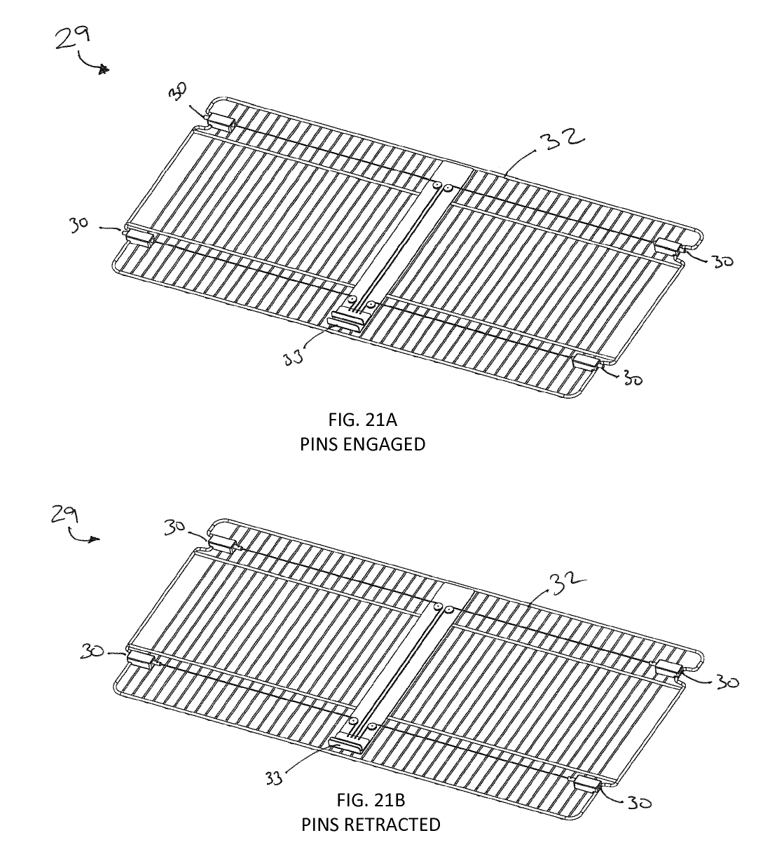

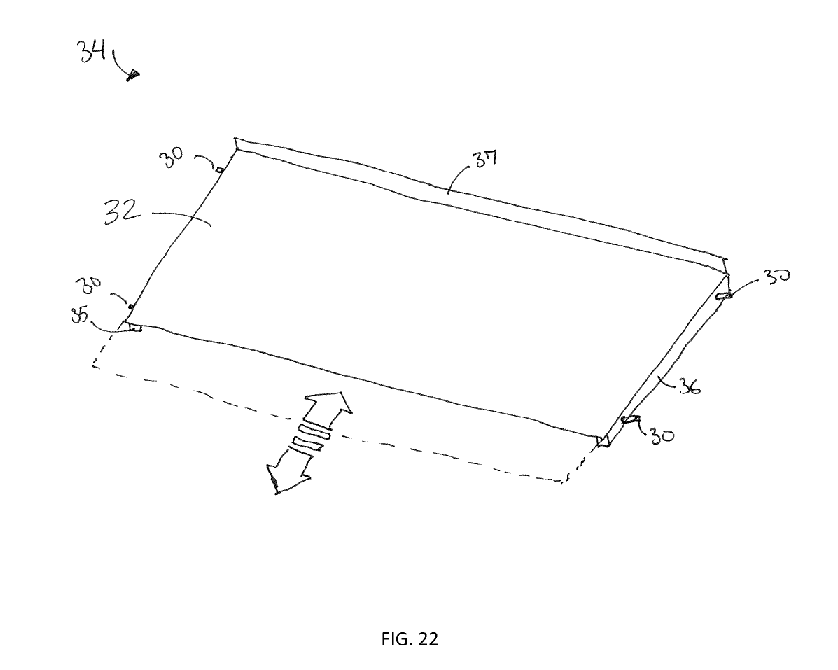

Referring to FIG. 22, a further embodiment of a containment device 13 may comprise a horizontal support surface 34, as disclosed above, that further comprises a left side extensible slide 35 and a right side extensible slide 36 configured such that the vented planar surface 32 is coincident with the extensible portions of the left and right extensible slides 35, 36, and the non-extensible portions of the left and right extensible slides are configured to accept the at least one retractable lock pin 30. This embodiment provides for a vented horizontal support surface that is vertically adjustable and can extend horizontally through the containment device aperture 9 for ease of loading and unloading of the load. An embodiment of this extensible shelf may also include a raised structure 37 along the back edge of the planar surface 32 to ensure that none of the load can fall off of the back edge.

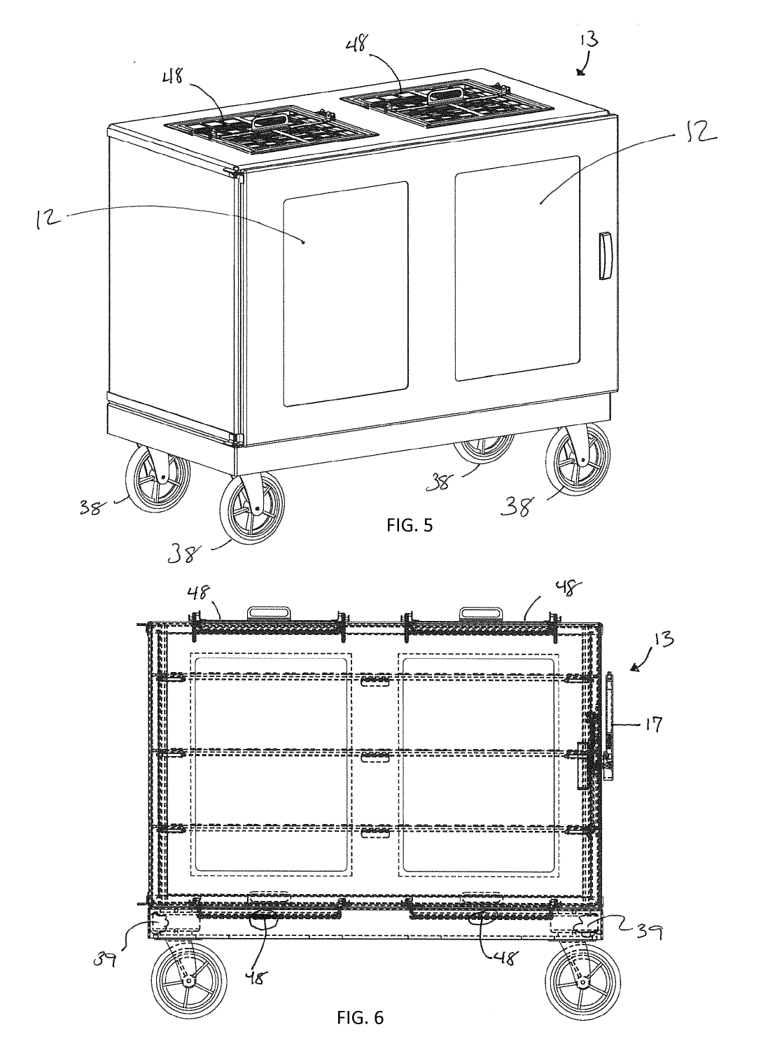

Referring to FIG. 5, yet another embodiment of a containment device 13 may further comprise a plurality of wheels 38 attached to the base, or floor 2, of the device 13 for ease in transferring the device 13 from one surface to another. Referring to FIG. 6, embodiments of the containment device 13 may incorporate a plurality of wheels 38 with shock absorbers 39 configured to absorb shocks and vibrations during transport in order to keep the load from rattling and moving, which may damage the load.

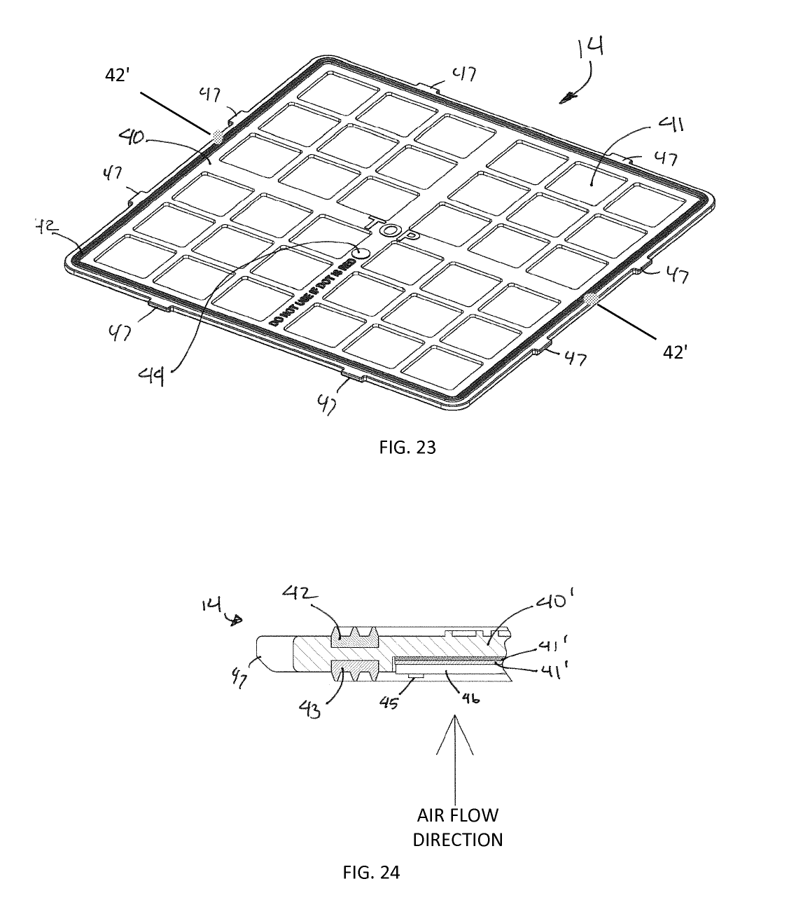

In FIG. 23, a filter apparatus 14 in accordance with an embodiment of the present invention may comprise a vented frame 40 with a top surface, a bottom surface, and an outer perimeter, an at least one layer of steam-permeable particulate filter material 41 fixedly attached to the vented frame bottom surface, a first compliant surface 42 coincident with the vented frame top surface along the vented frame outer perimeter configured to seal out microorganisms, prevent the migration of steam, and prevent the pooling of condensation, and a second compliant surface 43 coincident with the vented frame bottom surface along the vented frame outer perimeter configured to seal out microorganisms, prevent the migration of steam, and prevent the pooling of condensation.

Further embodiments of a filter apparatus 14 may include a single use indicator 44, such as a label or other indicator that is activated and, for example, changes color when exposed to any of the environmental conditions present in the sterilization cycle.

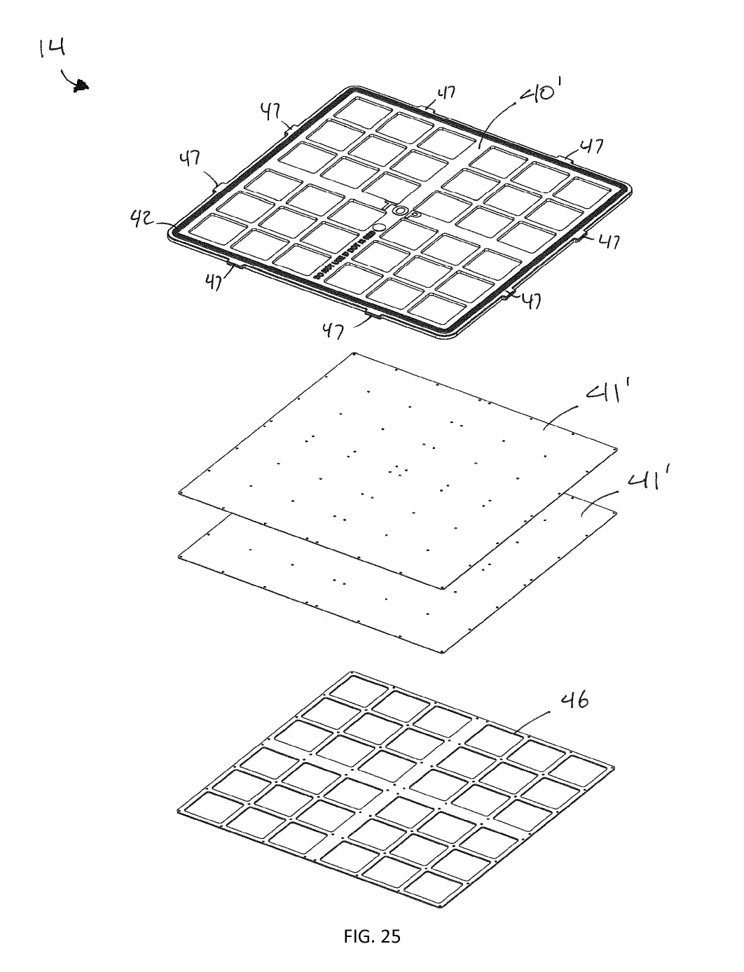

Referring to FIGS. 24, 25 and 26A, an embodiment of a filter apparatus 14 may comprise a vented frame 40′ with a top surface, a bottom surface, an outer perimeter and a one or more posts 45′ (See FIGS. 26A and 26B) protruding perpendicular to the bottom surface, an at least one layer of steam-permeable particulate filter material 41′ perforated with one or more holes 45 (FIG. 26B) configured to accept the one or more posts 45′ protruding perpendicular to the vented frame bottom surface (FIG. 26C), a vented filter backup plate 46 with a top surface, a bottom surface, an outer perimeter, and one or more holes configured to accept one or more posts 45 protruding perpendicular to the vented frame 40′ bottom surface, a first compliant surface 42 coincident with the vented frame top surface along the vented frame outer perimeter configured to seal out microorganisms, prevent the migration of steam, and prevent the pooling of condensation, a second compliant surface 43 coincident with the vented frame bottom surface along the vented frame outer perimeter configured to seal out microorganisms, prevent the migration of steam, and prevent the pooling of condensation, and a single use indicator configured to be activated by the sterilization cycle, wherein the vented filter backup plate is fixedly attached to the vented frame by permanently affixing the vented frame posts to the vented filter backup plate.

The manufacturing method employed to permanently affix the vented frame posts to the vented filter backup plate may include ultrasonic welding, press-fit/friction, mechanical snaps, chemical boding, or heat deformation.

Referring to FIG. 26B, a further embodiment of the filter apparatus 14 may contemplate that the vented frame posts 45′ end in a sharp point, thereby circumventing the need for mating holes in the filter media. As illustrated in FIG. 26C, any of the previous embodiments may further comprise a vented frame with one or more protrusions 47 extending out from the outer perimeter.

Any of the previous embodiments may further comprise channels and/or drain holes 42′ in the vented frame, optimally located between the compliant surface (seal) and the outer perimeter in order to prevent the pooling of condensation after the sterilization cycle. See FIG. 23.

An embodiment of the filter apparatus 14 may comprise a retro-fit kit whereby an embodiment of the filter apparatus 14, as described above with an integral gasket seal, may be used in a sheet-filtered containment device by first choosing an appropriately sized filter apparatus 14, removing the existing containment device's sheet filter gasket seal, inserting the filter apparatus 14 in lieu of the sheet filter media, and closing the filter retention door as indicated by the manufacturer.

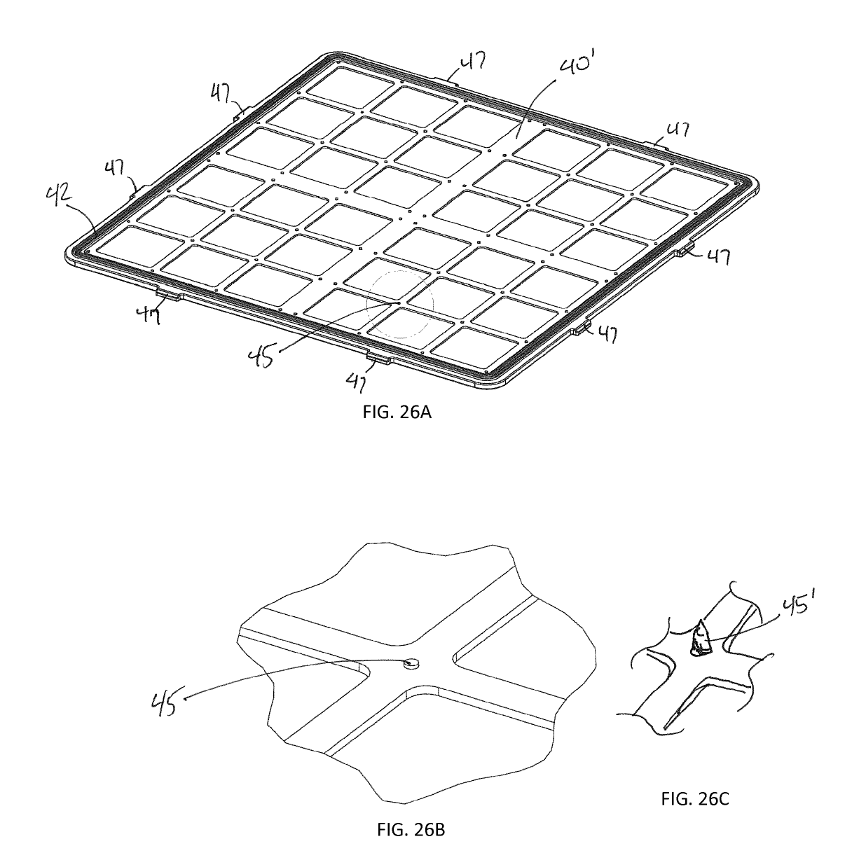

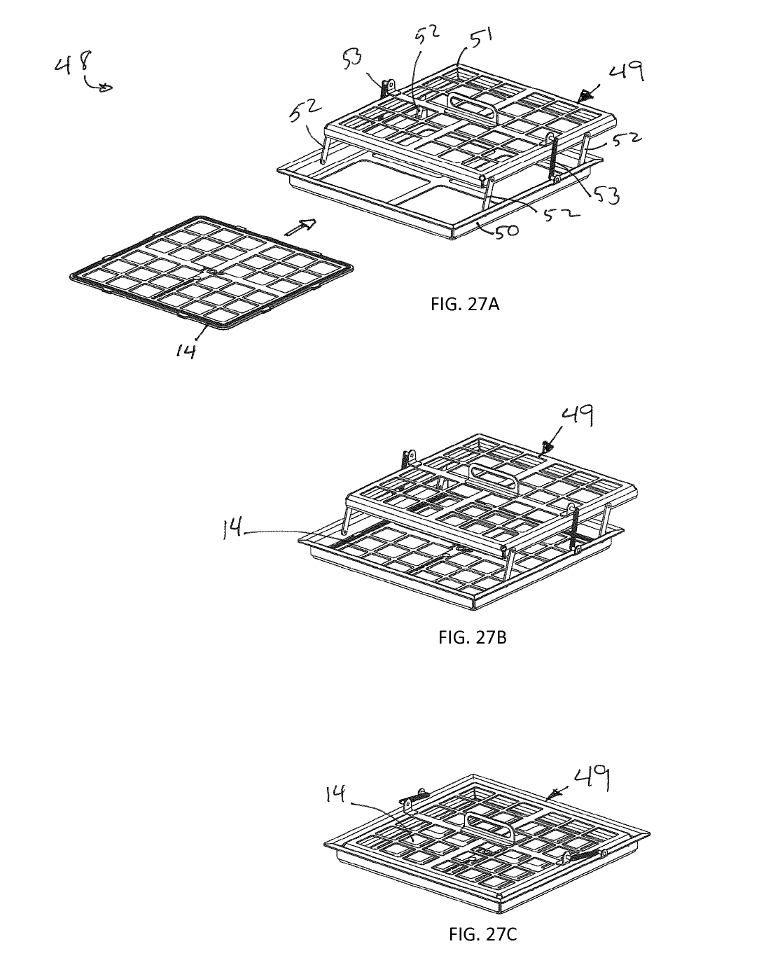

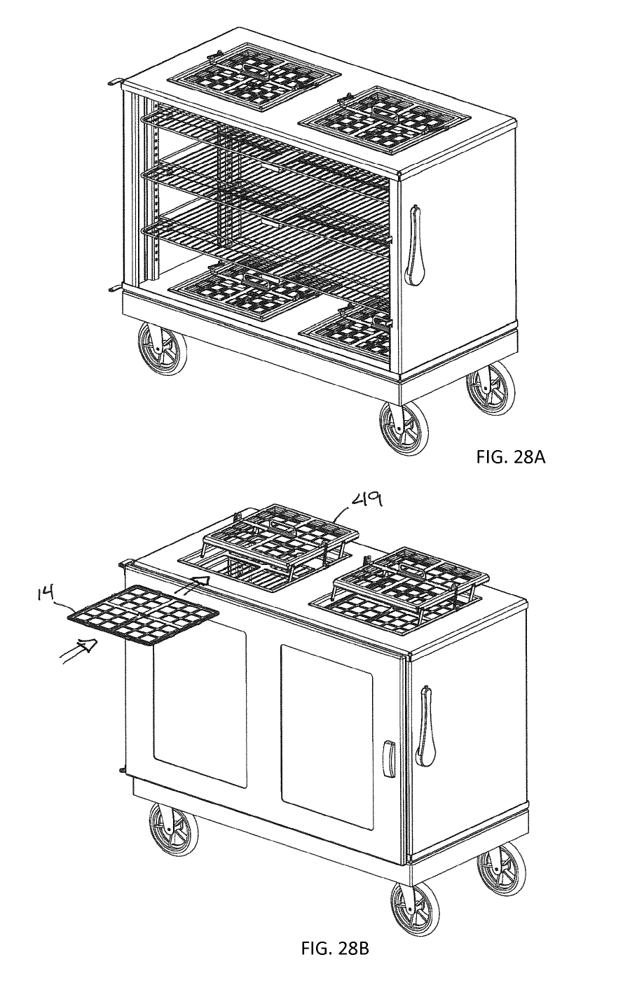

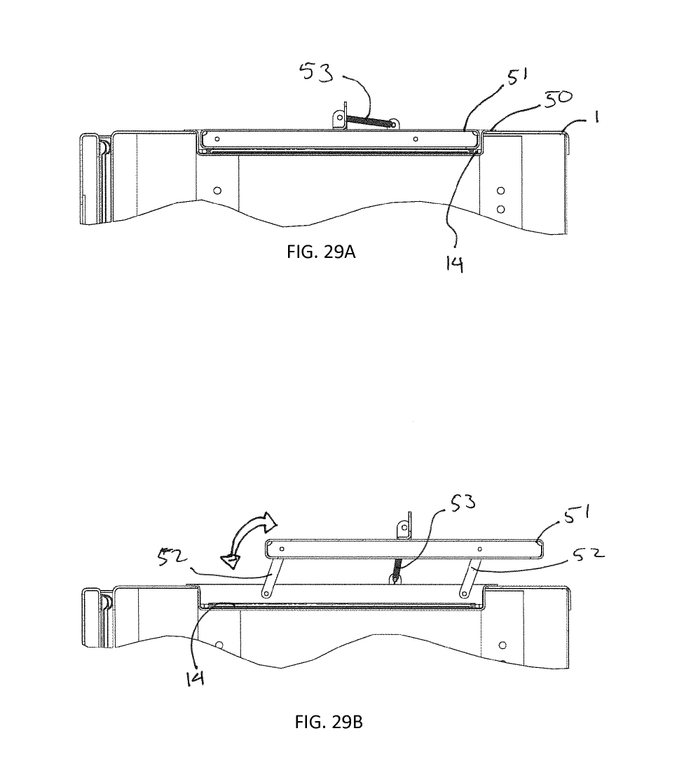

Referring to FIG. 27A, a filter system 48 may comprise a filter apparatus 14 in accordance with any of the previously discussed embodiments in combination with a filter retainer 49 (FIG. 27A, FIG. 27B, and FIG. 27C). The filter retainer 49 secures the filter apparatus 14 to the containment device 13 in order to allow sterilant to penetrate the device 13 during the sterilization cycle and to prevent microbial intrusion after the sterilization cycle.

Referring to FIG. 27A, an embodiment of a filter retainer 49 may comprise a filter-retention frame 50 defining an opening in the containment device 13, coincident with the containment device roof and floor void areas, with retention features configured to accept a filter apparatus 14, a filter clamp frame 51, a linkage mechanism 52 connecting the filter-clamp frame 51 to the filter-retention frame 50, and an extensible element 53 configured to maintain the door up in the open position (FIG. 27B) and down in the closed position (FIG. 27C).

Another embodiment of a filter retainer 49 may comprise a filter-retention frame defining an opening in the containment device 13, coincident with the containment device roof and floor void areas, with retention features configured to accept a filter apparatus 14, a one or more filter snap features configured to vertically hold the filter apparatus 14 in place, and a vented panel connected to and perpendicular to the filter-retention frame configured to prevent the filter apparatus 14 from being accessible to by the exterior of the containment device 13.

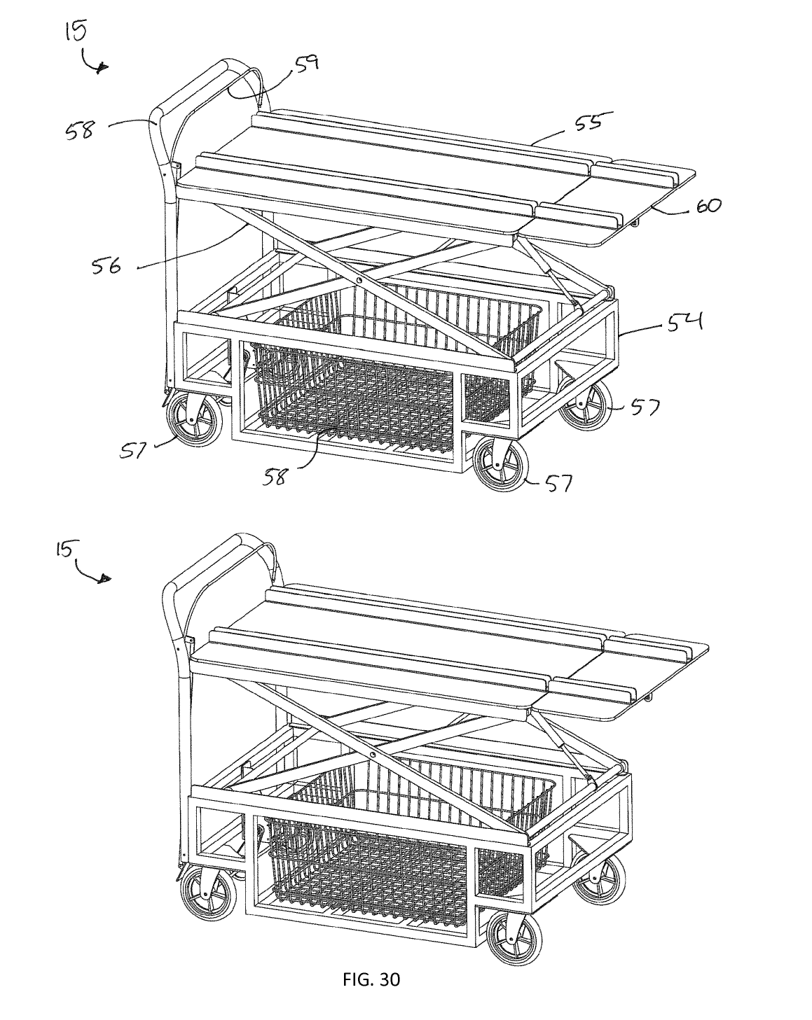

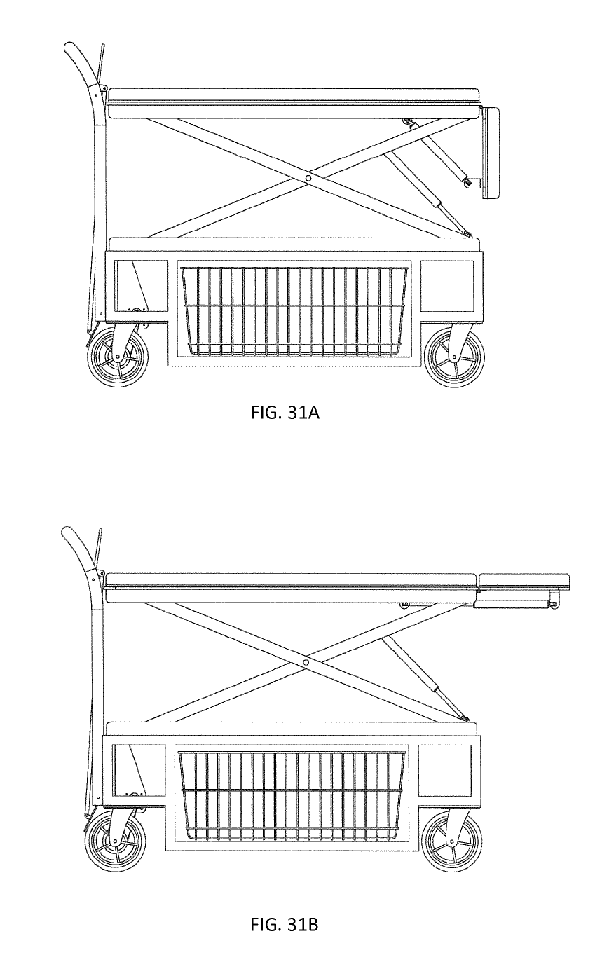

Referring now to FIGS. 30-35, a transport cart 15 in accordance with an embodiment of the present invention may comprise a first structural frame 54 (base), a second structural frame 55 (cabinet loading surface), a powered mechanism 56 for raising and lowering second structural frame (cabinet loading surface 55) relative to the first structural frame (base), a plurality of wheels 57, a braking mechanism configured to arrest wheel rotation, a drive mechanism configured to assist wheel rotation, a handle 58, a brake lever 59 configured to actuate the braking mechanism, and a hinged autoclave interface 60.

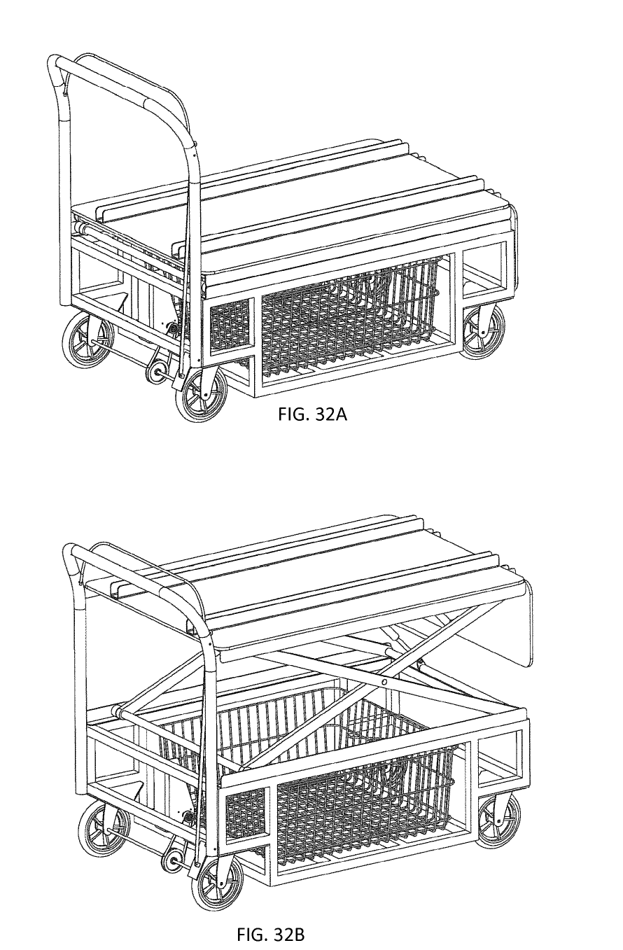

Referring to FIG. 32A, an embodiment of a transport cart 15 may have an upper platform configured to accept a containment device 13, a base, a handle, and a sterilization chamber interface 60 for facilitating entry into, or coupling with, a sterilization chamber such as an industrial autoclave. The upper platform of the transport device 14 may be configured to be vertically adjustable with respect to the base and the base may be configured with a plurality of wheels and a motorized drive system. FIG. 32B shows the vertical adjustment mechanism of the transport cart 15 of 32A raising the second structural frame 55.

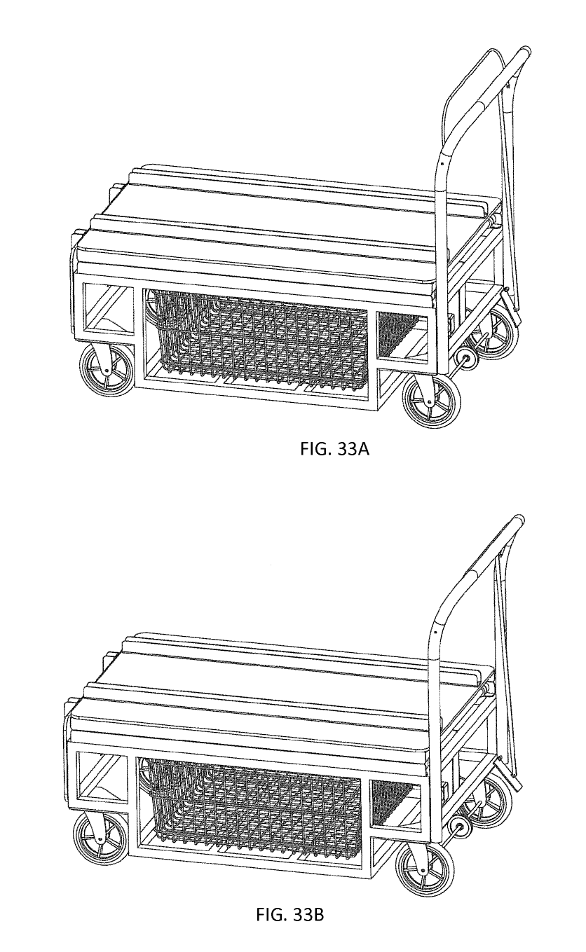

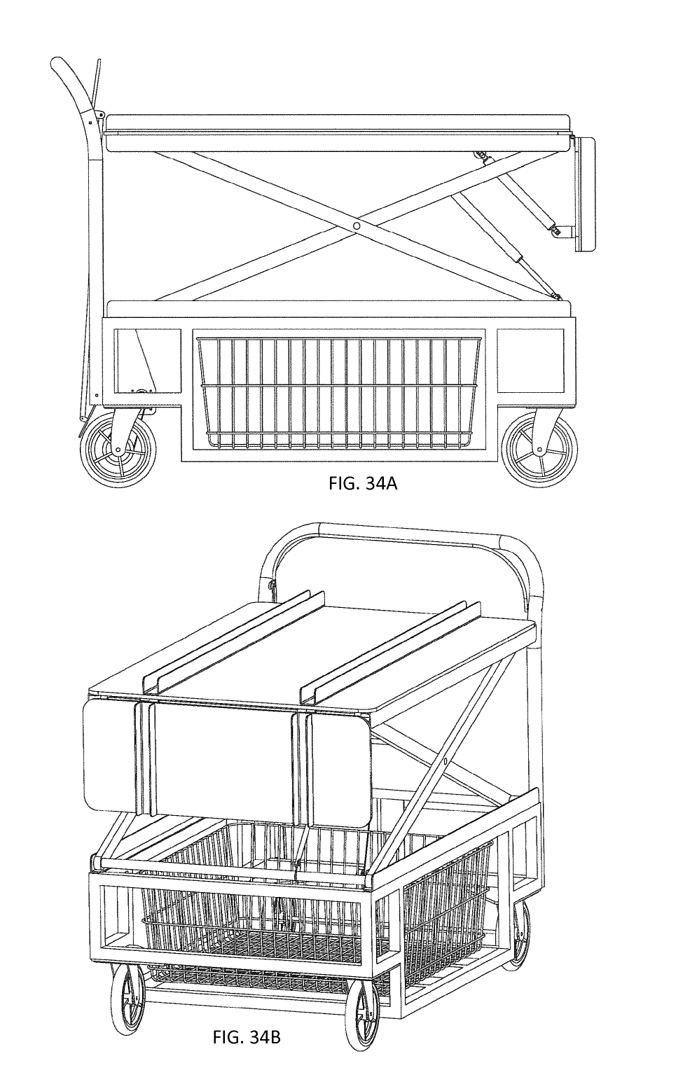

Referring now to FIGS. 33A and 33B, the handle may be configured to accept an input from an operator and only allow for the transport cart 15 to move when the operator applies the input. FIG. 33A shows the handle in the locked position, activating the braking mechanism, thus preventing the cart 15 from moving. The handle remains in the locked position until engaged by the operator. FIG. 33B shows the handle in the engaged position, releasing the braking mechanism and allowing the cart to be moved.

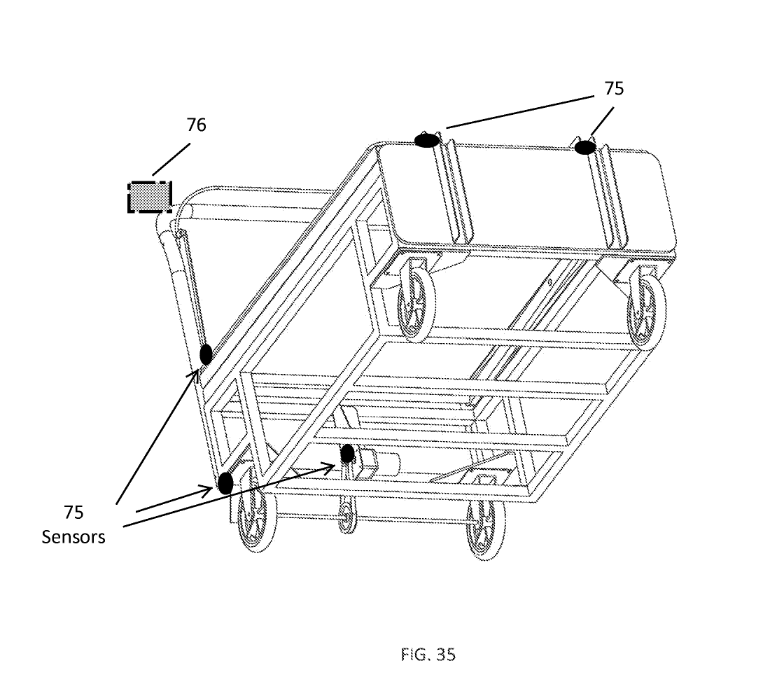

Referring now to FIGS. 34A and 34B, the sterilization chamber interface 60 coupled with the transport cart 15 is configured to be deployed and stowed by an operator of the transport cart 15. When in the open position, the interface 60 facilitates entry of the cabinet 13 into an autoclave such as an industrial autoclave in a hospital. When not in use, the interface 60 can be stowed such that the interface 60 does not extend beyond the cart 15. FIG. 34A shows a side view of the transport cart 15 with the hinged interface 60 in a stowed position FIG. 34B shows an oblique view of the stowed interface 60.

Referring now to FIG. 35, an embodiment of a transport cart 14 further comprises mechanical, electrical, or electromechanical sensing apparatus 75 configured to detect when the transport cart 15 is unloaded, loaded, docking, or undocking. Other use case scenarios may also be contemplated such as storage and transport. One skilled in the art will recognize that various sensing apparatus 75 may be employed such as electrical sensors or mechanical levers, and no particular limitation is implied herein except as stated in the claims. The transport cart 15, based upon input from the sensing apparatus 75, would, for example, and by means of illustration only, know when to release and deploy the autoclave interface 60, lock the containment device 13 for transport, and unlock the containment device 13 for loading into an autoclave. The transport cart 15 may employ various indicators 76 configured to visually convey the state of the various sensing apparatus 75.

Embodiments of the transport cart 15 may be configured to maintain the sterile field when unloading as well as maintaining an appropriate ergonomic height for loading and transporting by raising and lowering the second structural frame 55 as required. Embodiments of the transfer cart 15 may include a removable storage basket 58 in the base.

Yet other embodiments of the transfer cart 15 may further incorporate a plurality of wheels 57 configured to absorb shocks and vibrations during transport in order to keep the load from rattling and moving, which may damage the load.

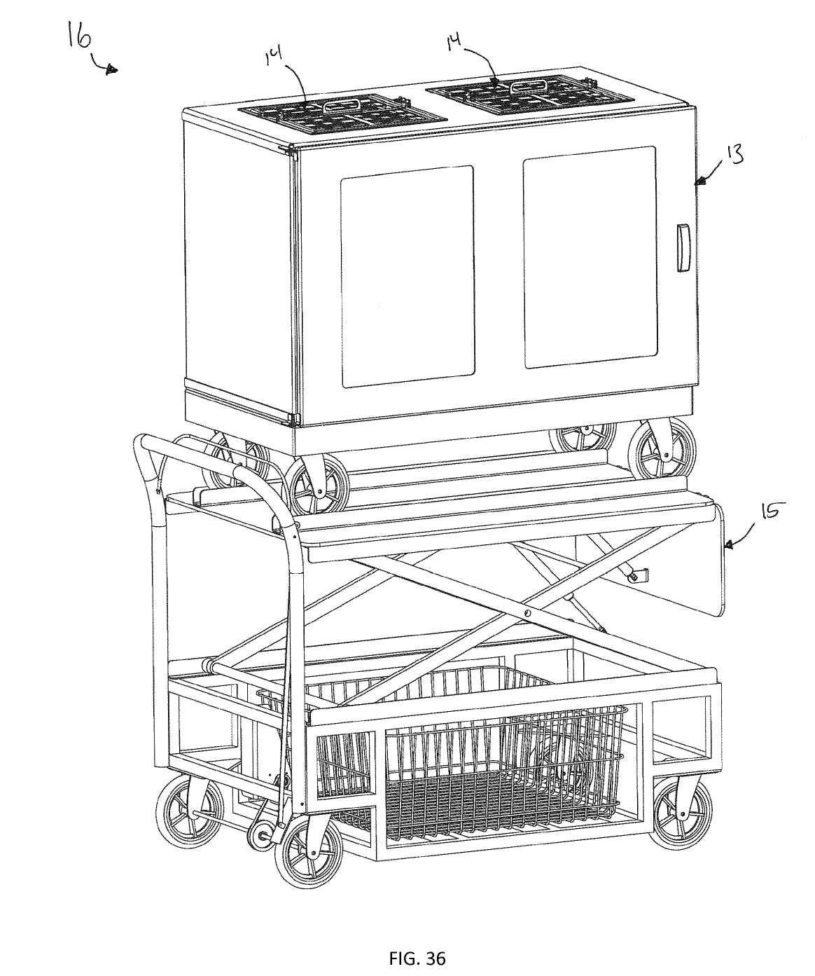

Referring to FIG. 36, an autoclavable sterilization, storage, and transport system 16 for reusable medical devices in accordance with an embodiment of the present invention may comprise a containment device 13, at least one filter apparatus 14, and a transport cart 15. The containment device 13 may further include: a roof 1, a floor 2, a left side 3, a right side 4, and a back side 5 defining a containment device interior 6, a containment device exterior 7, and a containment device aperture 8. The containment device interior 6 may be configured to accept a load for sterilization. The containment device aperture 8 may be configured to sealably accept an at least one containment device door. The roof 1 and floor 2 may each be configured to sealably accept an at least one filter apparatus 14.

The at least one filter apparatus 14 may have an at least one layer of steam-permeable filtration material, a support frame, a retention frame, an integral seal, and a single use indicator. The transport cart 15 may have an upper platform configured to accept a containment device 13, a base, a handle, and an autoclave interface 60. The upper platform of the transport device 15 may be configured to be vertically adjustable with respect to the base and the base may be configured with a plurality of wheels 57 and a motorized drive system. The handle may be configured to accept an input from an operator and only allow for the transport cart 15 to move when the operator applies the input. The autoclave interface 60 of the transport cart 15 may be configured to be deployed and stowed by the operator.

It will be appreciated that the devices, apparatus, and systems described above are set forth by way of example and not of limitation. Numerous variations, additions, omissions, and other modifications will be apparent to one of ordinary skill in the art.

While particular embodiments of the present invention have been shown and described, it will be apparent to those skilled in the art that various changes and modifications in form and details may be made therein without departing from the spirit and scope of the invention as defined by the following claims. The claims that follow are intended to include all such variations and modifications that might fall within their scope, and should be interpreted in the broadest sense allowable by law.

In light of the foregoing description, it should be recognized that embodiments in accordance with the present invention can be realized in numerous configurations contemplated to be within the scope and spirit of the claims. Additionally, the description above is intended by way of example only and is not intended to limit the present invention in any way, except as set forth in the claims.

1. A transport apparatus for a sterilization cabinet, said transport apparatus comprising:
a base structural frame comprising:
a plurality of wheels;
shock absorbers coupled with the plurality of wheels;
a braking mechanism; and
a motorized drive mechanism;
a platform configured to accept the sterilization cabinet, wherein said platform is vertically adjustable relative to the base structural frame;
a vertical adjustment mechanism coupled with the platform for raising and lowering said platform relative to the base structural frame;
a hinged sterilization chamber interface coupled with the platform; and
a handle.

2. The transport apparatus of claim 1 further comprising sensing apparatus configured to detect when the transport apparatus is unloaded, loaded, docking, and undocking.

3. The transport apparatus of claim 1 wherein the handle accepts input from an operator of said transport apparatus and allows said transport apparatus to move only when the operator applies said input.

4. The transport apparatus of claim 1 wherein the base structural frame further comprises a removable storage basket.

5. A sterilization cabinet comprising:
an autoclavable rigid containment device with at least one filtered vent, said autoclavable rigid containment device comprising:
a floor and a roof, each comprising a flat surface portion configured to accept at least one filter apparatus;
an interior configured to accept a sterilization load;
an exterior;
an aperture comprising an aperture seal configured to sealably accept an at least one autoclavable rigid containment device door; and
the at least one autoclavable rigid containment device door comprising at least one viewing window allowing inventory and identification of the sterilization load.

6. The sterilization cabinet of claim 5 wherein the at least one autoclavable rigid containment device door comprises a door latch mechanism with a rotating arm actuating a two-part action, said two-part action comprising:
a first action opening the at least one filtered vent, equalizing air pressure between the interior and an outside environment; and
a second action occurring once the air pressure is equalized, said second action releasing a latch from the aperture seal, and breaking said aperture seal, providing aseptic presentation of the sterilization load.

7. The sterilization cabinet of claim 5 wherein the roof and floor further comprise a perimeter lip around the flat surface portion, and at least one void area accepting of the at least one filter apparatus.

8. The sterilization cabinet of claim 5 further comprising the at least one filter apparatus sealably secured to the at least one vent.

9. The sterilization cabinet of claim 8, wherein the at least one filter apparatus is a vented frame comprising:
a top surface;
a bottom surface;
an outer perimeter;
an at least one layer of steam-permeable particulate fiber material fixedly attached to the bottom surface;
a first compliant surface coincident with the top surface along an outer perimeter of said top surface, sealing the filter apparatus against microorganisms, preventing migration of steam, and preventing pooling of condensation; and
a second compliant surface coincident with the bottom surface along an outer perimeter of said bottom surface, sealing the filter apparatus against microorganisms, preventing migration of steam, and preventing pooling of condensation.

10. The sterilization cabinet of claim 9, wherein the at least one filter apparatus further comprises channels disposed between the first compliant surface and the outer perimeter, preventing pooling of condensation after a sterilization cycle.

11. The sterilization cabinet of claim 9, wherein the at least one filter apparatus further comprises channels disposed between the second compliant surface and the outer perimeter, preventing pooling of condensation after a sterilization cycle.

12. The sterilization cabinet of claim 9, wherein the at least one filter apparatus comprises a filter retainer securing said at least one filter apparatus to the rigid containment device, allowing sterilant to penetrate said rigid containment device.

13. The sterilization cabinet of claim 5 wherein the interior further comprises at least one vented horizontal support surface for placing the load for sterilization.

14. The sterilization cabinet of claim 13 wherein the at least one vented horizontal support surface comprises an extensible shelf that is vertically adjustable.

15. The sterilization cabinet of claim 14 wherein the at least one vented horizontal support surface is horizontally adjustable, wherein said extensible shelf can extend horizontally through the aperture for ease of loading and unloading the sterilization load.

16. A filter system for a sterilization cabinet comprising:
at least one filter apparatus securably sealed to a vent in the sterilization cabinet allowing sterilant to penetrate the sterilization cabinet device during a sterilization cycle preventing microbial intrusion after said sterilization cycle, said at least one filter apparatus comprising:
at least one layer of steam-permeable filtration material;
a support frame;
a filter retainer securing the at least one filter apparatus to the sterilization cabinet;
a linkage mechanism connecting the support frame to the filter retainer;
an integral seal; and
a single use indicator.

17. The filter system of claim 16 wherein the support frame is a vented frame comprising:
a top surface;
a bottom surface on which the at least one layer of steam-permeable particulate filter material is fixedly attached; and
an outer perimeter.

18. The filter system of claim 17 wherein the integral seal comprises:
a first compliant surface along the outer perimeter of the top surface; and
a second compliant surface along the outer perimeter of the bottom surface.

19. The filter system of claim 18 further comprising channels disposed between the integral seal and the outer perimeter, said channels preventing condensation after the sterilization cycle.

20. The filter system of claim 16 wherein the single use indicator changes color when exposed to sterilant.
